(19) United States
(12) Patent Application Publication
(10) Pub. No.: US 2020/0200719 A1
(43) Pub. Date: Jun. 25, 2020

(54) Process For The Characterization Of Glatiramer Acetate

(71) Applicant: Alembic Pharmaceuticals Limited, Vadodara (ID)

(72) Inventors: Navya Sama, Hyderabad (IN); Tumpa Das, Hyderabad (IN); Srinivas Suryapeta, Hyderabad (IN); Deepak Mogulla, Hyderabad (IN); Sourabh Kumar, Hyderabad (IN); Praveer Gupta, Hyderabad (IN)

(21) Appl. No.: 16/643,692

(22) PCT Filed: Sep. 5, 2018
(86) PCT No.: PCT/IB2018/056770

§ 371 Date: Mar. 2, 2020

(30) Foreign Application Priority Data
Sep. 7, 2017 (IN) IN201721031750

(51) Int. Cl. G01N 30/74; G01N 33/68; G01N 30/72

(52) U.S. Cl. CPC G01N 30/74; G01N 2560/00; G01N 30/7266; G01N 33/6848

(57) ABSTRACT

The present invention is directed to process for characterization of Glatiramer acetate. The present invention further relates to process for characterization of Glatiramer acetate using size exclusion chromatography coupled with ultra performance liquid chromatography (SEC-UPLC) and high resolution mass spectrometry (HRMS). The present invention further provides statistical methods for characterizing and classifying Glatiramer acetate.

### FIELD OF THE INVENTION

The present invention is directed to process for characterization of Glatiramer acetate. The present invention further relates to process for characterization of Glatiramer acetate using size exclusion chromatography coupled with liquid chromatography such as ultra-performance liquid chromatography (SEC-UPLC) and high resolution mass spectrometry (HRMS). The present invention further provides statistical methods for characterizing and classifying Glatiramer acetate.

### BACKGROUND OF THE INVENTION

Glatiramer acetate is a synthetic polypeptide analog of myelin basic protein (MBP), which is a natural component of the myelin sheath. Glatiramer acetate is approved for the treatment of patients with relapsing-forms of multiple sclerosis.

Glatiramer acetate consists of acetate salts of synthetic polypeptides, containing four naturally occurring amino acids: L-glutamic acid, L-alanine, L-tyrosine and L-lysine with an average molar fraction of 0.141, 0.427, 0.095 and 0.338 respectively. The average molecular weight is 5,000 to 9,000 Daltons.

Glatiramer acetate is prepared by polymerization reaction and since polymerization reactions are of stochastic nature, the Glatiramer acetate obtained by this process varies in its molecular weight resulting in variations in molecular weight distribution. Hence, Glatiramer acetate poses significant challenge in the characterization of the molecule due to lot-to-lot variations attributed to its complex nature. It is necessary to obtain Glatiramer acetate with the desired average molecular weight or molecular weight distribution in order to ensure its safety and efficacy for use in pharmaceutical products.

WO2000027417, WO2012016042 and WO2012123959 disclose process for the characterization of Glatiramer acetate using molecular weight markers.

WO2007127977 and WO2013009885 disclose analysis of dimethylamide and related fragments.

WO2010129851 (IN4606/KOLNP/2011) discloses use of mass spectroscopy to analyze enzyme-digested peptide fragments.

WO2013009864 discloses process for the characterization of Glatiramer acetate using measurement of alpha helical content and random coil content.

WO2015044876 (IN201647013785) discloses process for the characterization of Glatiramer acetate using trifluoroacetyl glatiramer as molecular weight marker.

WO2016172855 discloses process for the characterization of Glatiramer acetate using anion exchange liquid chromatography, cation exchange chromatography or reverse phase liquid chromatography.

WO2016176649 discloses process for the characterization of Glatiramer acetate as function of hydrophobicity using multi angle laser light scattering (MALLS) technique.

US20170089916 discloses process for the characterization of Glatiramer acetate by using size exclusion chromatography associated with Multi-Angle Light Scattering (SEC-MALS).

Most of the prior art processes disclosed above use molecular weight markers. However, this approach has shortcomings as molecular weight markers with narrow molecular weight distribution will be different from the Glatiramer acetate mixture. And hence, the molecular weight distribution assessed by use of molecular weight markers can only be relative, but not absolute molecular weight distribution. Furthermore, molecular weight markers are difficult to prepare synthetically or by recombinant technology and are expensive.

Another technique disclosed in prior art processes above is Size-exclusion chromatography associated with MALS. SEC-MALS involves use of more than one detection systems and requires stabilization of each detection system which is tedious task. Furthermore, SEC-MALS technique uses slices of the peak to derive molecular weight. Since, the elution pattern of Glatiramer acetate is too complex, it is difficult to have similar dispersity at each slice. SEC-MALS technique produces the stoichiometric average molecular weight, however it is very difficult to predict whether a narrow molecular weight distribution is actually achieved. SEC-MALS only produces an aggregate folding pattern, while single folding pattern of each peptide cannot be established which leads to generation of false molecular weight data.

Another technique disclosed in prior art processes above is matrix assisted laser desorption ionization-time of flight mass spectrometry (MALDI-TOF MS). In MALDI-TOF each zone of slicing can be spotted on to the plate. However, for a complex molecule like Glatiramer acetate, the number of peptides in each slice, with their variable nature of polarity poses a difficult problem. It is known that sample preparation is one of the key points for MALDI. Since, each slice has possible different range of the polarities of Glatiramer peptides, the matrix suitability for the peptides is a very difficult choice.

Therefore, prior art processes for characterization of Glatiramer acetate always do not possess desired repeatability and reproducibility of molecular weight distribution determination. Hence there is a need to develop new robust analytical technique for characterization of Glatiramer acetate which can be applied routinely for quality control and/or batch release purposes.

The present inventors have surprisingly found out a process for characterization of Glatiramer acetate using SEC/HRMS method. The process for characterization of Glatiramer acetate according to present invention overcomes drawbacks associated with prior art process. The present invention further provides statistical methods for characterizing and classifying Glatiramer acetate.

### SUMMARY OF THE INVENTION

In one aspect, the present invention relates to a process for characterization of Glatiramer acetate using size exclusion chromatography coupled with liquid chromatography such as ultra performance liquid chromatography (SEC-UPLC) and high resolution mass spectrometry (HRMS).

In one aspect, the present invention relates to a process for characterization of Glatiramer acetate by identifying the variations in the molecular weight distribution pattern of Glatiramer acetate.

In one aspect, the present invention relates to identification of different molecular ions available within Glatiramer acetate using high resolution mass spectrometry (HRMS).

In one aspect, the present invention relates to a process for characterization of Glatiramer acetate using size exclusion chromatography coupled with liquid chromatography such as ultra performance liquid chromatography and UV detector (SEC-UPLC-UV).

In one aspect, the present invention relates to determination of the variation parameters using size exclusion chromatography coupled with liquid chromatography such as reverse phase liquid chromatography (SEC-RPLC).

In another aspect, the present invention relates to a process for characterization of Glatiramer acetate comprising:
a. subjecting a sample of Glatiramer acetate to size exclusion chromatography (SEC);
b. separating various fractions based on its size and subjecting the separated fractions through reverse phase chromatography to HRMS;
c. processing of electrospray ionized ion masses and obtaining deconvoluted masses for checking mass distribution pattern of Glatiramer acetate; and
d. Characterizing the Glatiramer acetate using statistical model.

In one aspect, the present invention relates to statistical methods for characterizing and classifying Glatiramer acetate.

In once aspect, the present invention relates to the variability evaluation through ion mobility mass spectroscopy.

### BRIEF DESCRIPTION OF THE DRAWINGS

FIG. 1 is an illustration of deconvoluted spectra of Copaxone V01821 (RLD) and the test samples (GLAT/001/044 & GLAT/001/084)

FIG. 2 is an illustration of Normal distribution curves for fraction 8.5-9 mins, representative fraction, for Copaxone V01821 (RLD) and the test samples (GLAT/001/044 & GLAT/001/084)

FIG. 3 is an illustration of scatter plot showing the standard deviations of masses observed in different lots of Copaxone and test samples. Table 3, 4 and 5 are representative tables for showing the mean and standard deviations for Copaxone V01821 (RLD) and the test samples (GLAT/001/044 & GLAT/001/084) respectively.

FIG. 4 is an illustration of Bar graph representing the distribution of masses in various mass categories shown in Table 6

FIG. 5 is an illustration of Molecular weight distribution of Glatiramer acetate using trend plots for PDI, Mw, Mn and Mz for different lots of Copaxone and test samples.

FIG. 6 is an illustration of mass determination of standard proteins using Unifi and in-house statistical models checked for confirming the accuracy of the novel protocol. It was followed by analyzing Copaxone and Test samples using the same protocol, the Tables 1 & 2 are based on the observations of the mentioned analyses.

FIG. 7 is an illustration of extremity of various parameters mass distribution parameters for different lots of Copaxone & test samples based on Tables 7 to 12.

FIG. 8 is an illustration of Heat Map showing distribution for Copaxone and test batches through IMMS

FIG. 9 is an illustration of Heat Map showing variations in mass distributions of Copaxone against Test samples through IMMS

FIG. 10 is an illustration of CD data of Copaxone vs. Test-samples

FIG. 11 is an illustration of NMR data of Copaxone vs. Test-samples

### BRIEF DESCRIPTION OF ABBREVIATIONS

CD: Circular Dichroism Spectroscopy
ESI: Electrospray Ionization
GMA: Glatiramer acetate
HRMS: High Resolution Mass Spectrometry
IMMS: Ion-Mobility Mass spectrometry
IMS QTof: Ion Mobility Quadrupole Time-of-flight Mass Spectrometry
IR: Infra Red spectroscopy
LC: Liquid Chromatography
MALDI-TOF-MS: Matrix Assisted Laser Desorption Ionization-Time of Flight Mass Spectrometry
MALS: Multi Angle Light Scattering
MALLS: Multi Angle Laser Light Scattering
MBP: Myelin Basic Protein
Mn: Number Average Molecular Weight
Mw: Weight Average Molecular Weight
Mz: Z Average Molecular Weight
MWD: Molecular Weight Distribution
NMR: Nuclear Magnetic Resonance spectroscopy
PDI: Poly Dispersity Index
RLD: Reference Listed Drug
RPLC: Reverse Phase Liquid Chromatography

SEC: Size Exclusion Chromatography
UPLC: Ultra Performance Liquid Chromatography
UV: Ultra Violet
Tof: Time of Flight
Std. dev.: Standard Deviation

### DETAILED DESCRIPTION OF THE INVENTION

It is to be understood that the invention is not limited in its application to the details set forth in the following description or exemplified by the Examples. The invention is capable of other embodiments or of being practiced or carried out in various ways. Also, it is to be understood that the phraseology and terminology employed herein is for the purpose of description and should not be regarded as limiting.

The present invention provides robust process for characterization of Glatiramer acetate which provides desired repeatability and reproducibility of molecular weight distribution determination.

In one embodiment, the present invention relates to a process for characterization of Glatiramer acetate using size exclusion chromatography coupled with liquid chromatography such as ultra performance liquid chromatography (SEC-UPLC) and high resolution mass spectrometry (HRMS).

In one embodiment, the present invention relates to a process for characterization of Glatiramer acetate by identifying the variations in the molecular weight distribution pattern of Glatiramer acetate.

In one embodiment, the present invention relates to identification of different molecular ions available within Glatiramer acetate using high resolution mass spectrometry (HRMS).

In one embodiment, the present invention relates to a process for characterization of Glatiramer acetate using size exclusion chromatography coupled with ultra performance liquid chromatography and UV detector (SEC-UPLC-UV).

In one embodiment, the present invention relates to determination of the variation parameters using size exclusion chromatography coupled with reverse phase liquid chromatography (SEC-RPLC).

In another embodiment, the present invention relates to a process for characterization of Glatiramer acetate comprising:
a) subjecting a sample of Glatiramer acetate to size exclusion chromatography coupled with UPLC;
b) collecting the fractions at different time points in SEC;
c) cubjecting the fractions through reverse phase chromatography coupled (with UV detector) with HRMS; and
d) obtaining molecular weight distribution pattern of Glatiramer acetate using Unifi software of waters system.

The size exclusion chromatography system as used herein includes components thereof known to the person skilled in the art, such as, one or more pumps for maintaining constant flow rate; column types for the molecular weight range of interest; and a detector system for detecting and/or quantifying the result. The mobile phase comprises a solvent and a portion of the protein, peptide, polypeptide, or fragment thereof disposed therein flows past a stationary phase. The stationary phase typically comprises finely divided, porous particles, such as microporous crosslinked agarose-based gels, modified polymethylmethacrylate gels, or porous silica. Detector system comprises mass concentration sensitive detector (e.g. refractive index detector) or molar concentration sensitive detector (e.g., ultraviolet light detector). Other methods include a density detector and an evaporative light-scattering detector.

In one embodiment, the present invention relates to the variability evaluation through ion mobility mass spectroscopy.

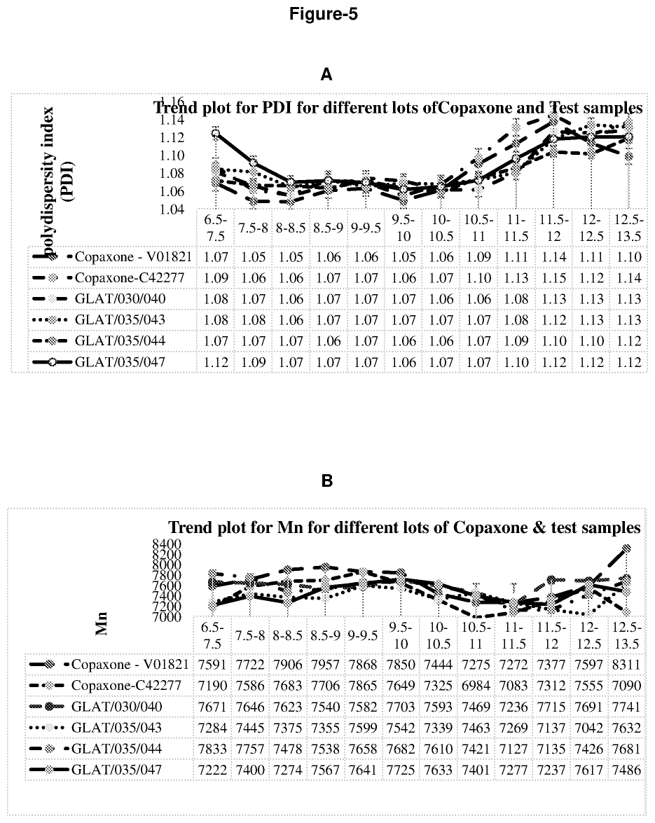

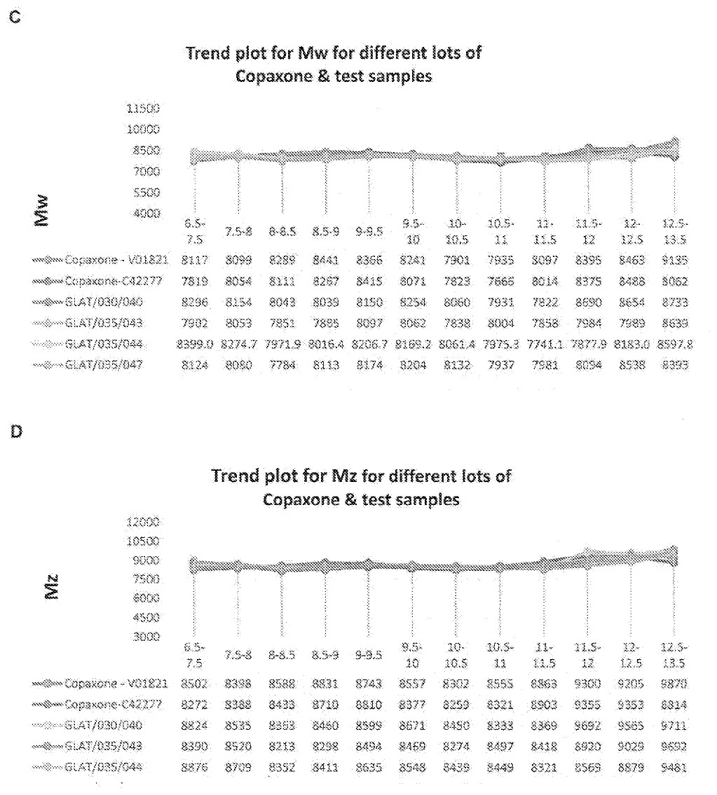

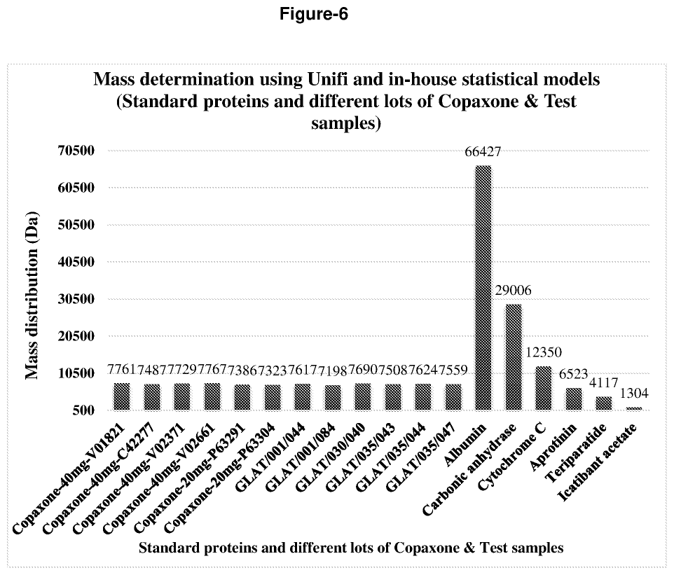

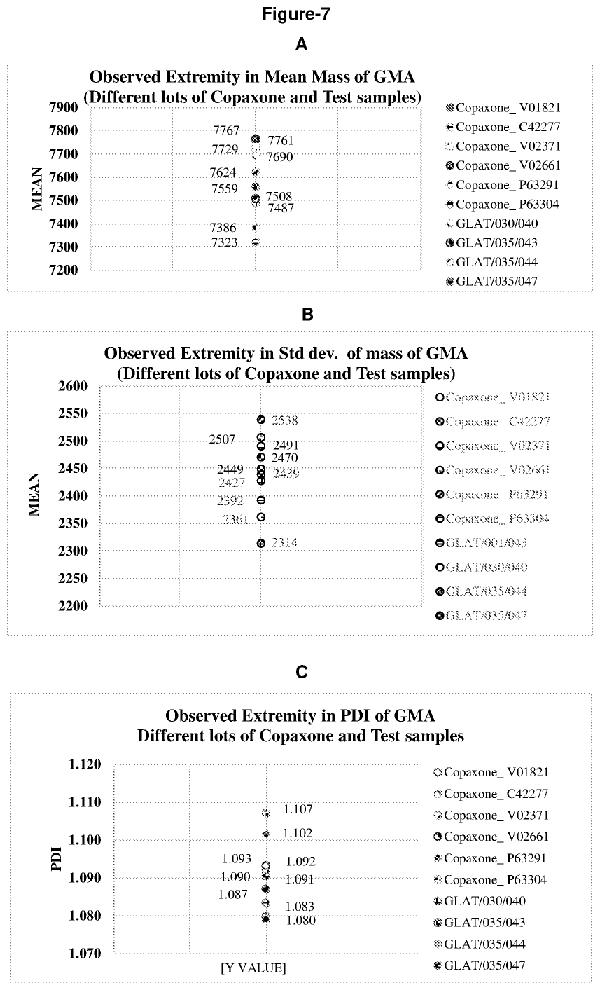

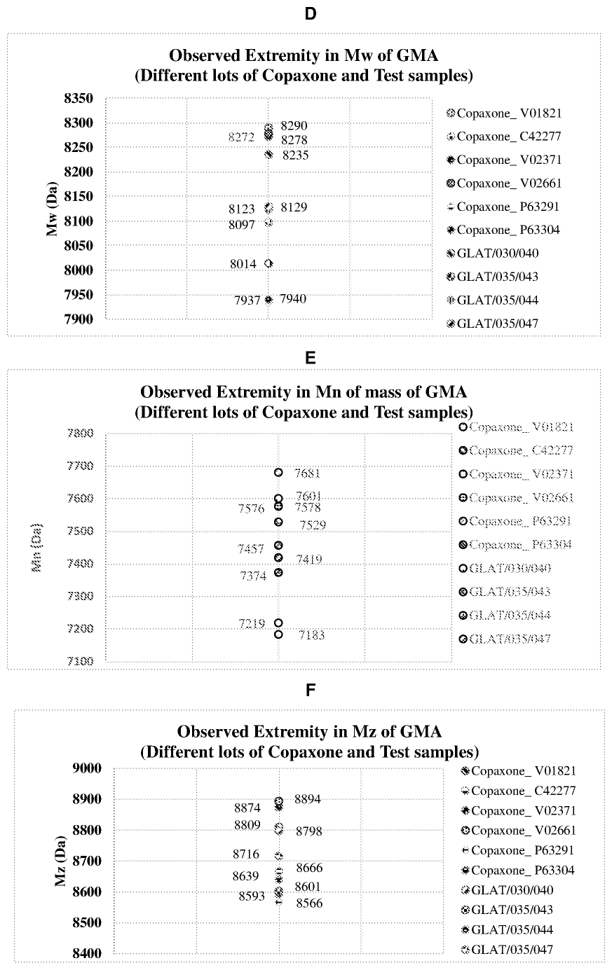

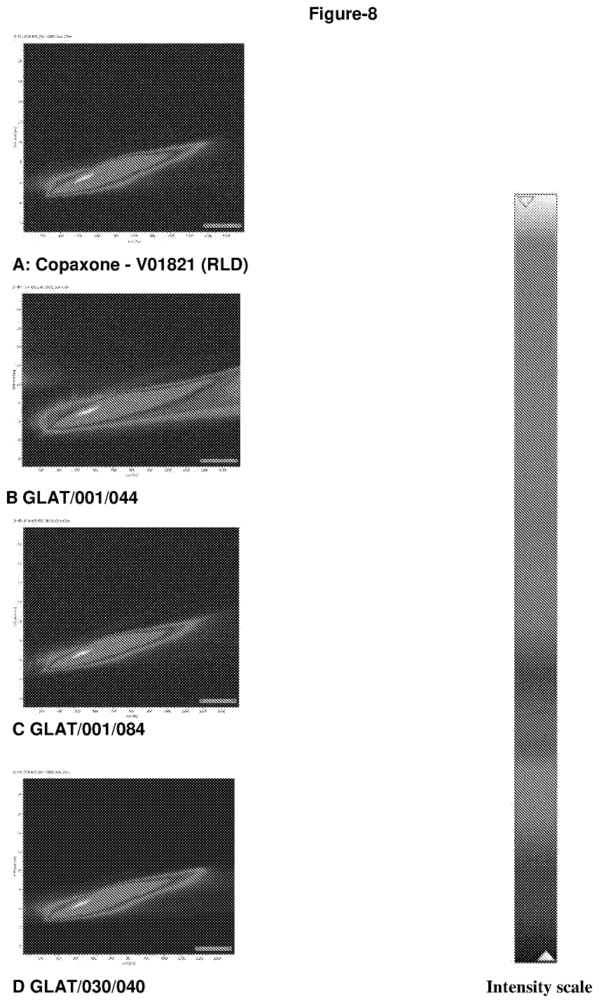

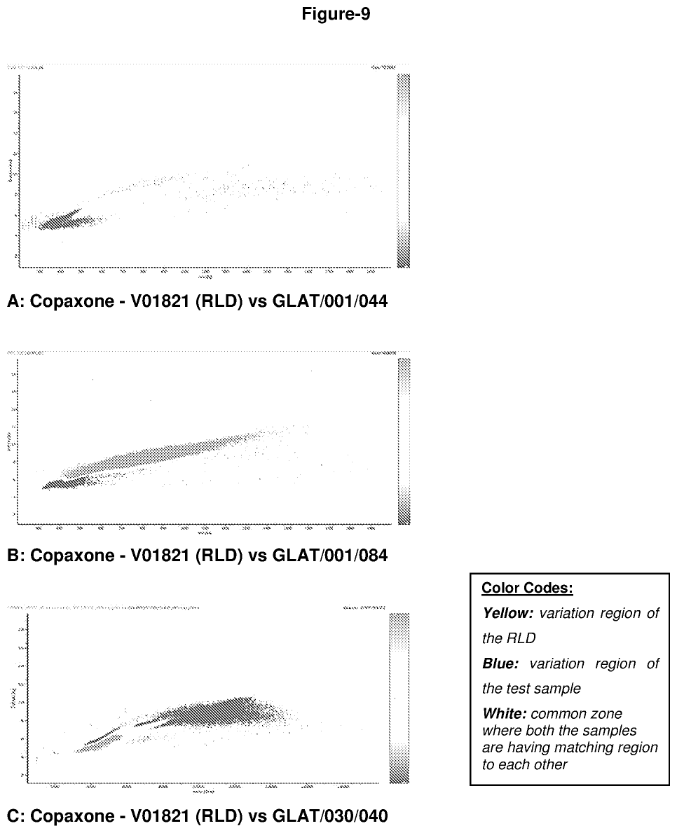

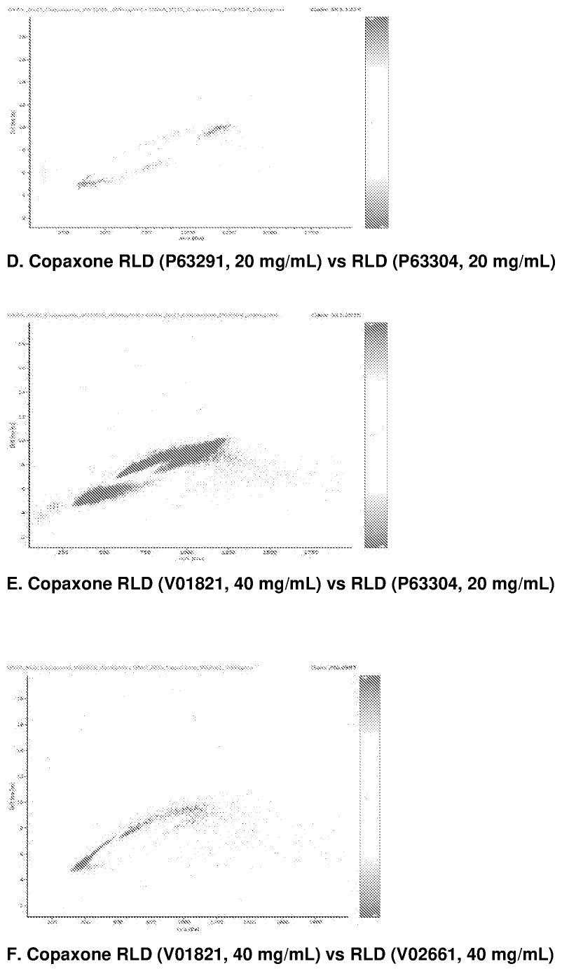

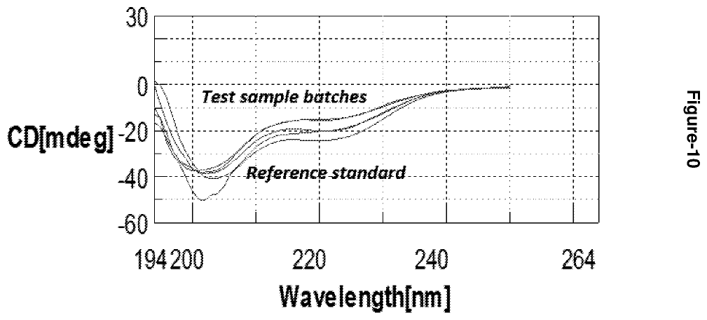

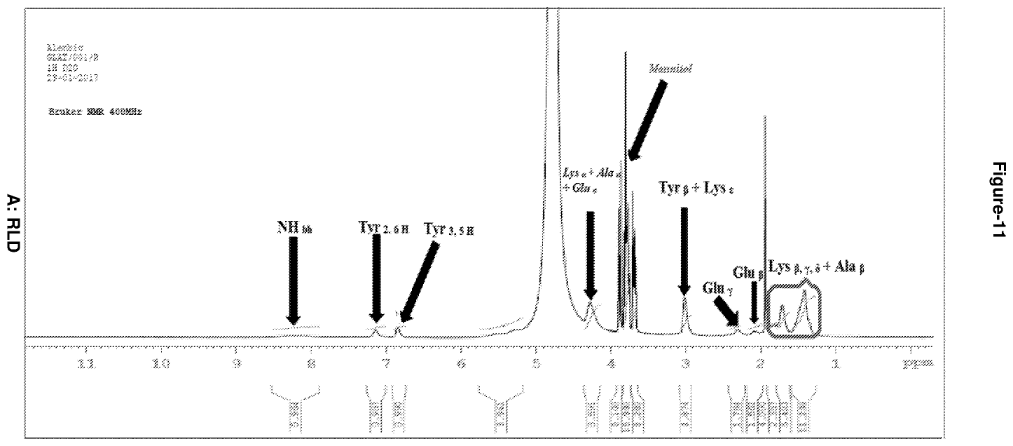

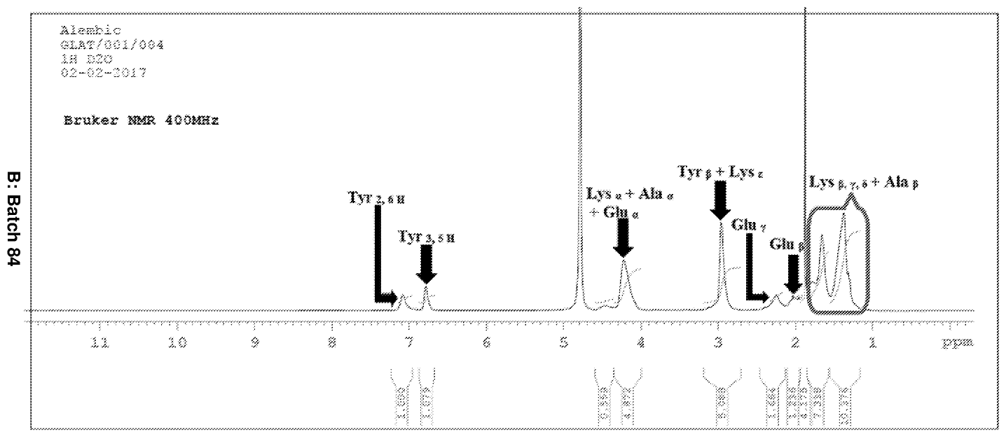

While performing SEC-UPLC, it is essential to ascertain variations within each slice in comparison with the reference sample. To check variability, each slice is mapped in 10 distinctive zones based on retention time and fractions of SEC eluent are collected and run on HRMS using reverse phase UPLC methodology. The reverse phase methodology is also useful in understanding the trend lines of the copolymer mass. Using the data obtained from the fractions collected after de-isotoping the spectrum, the molecular weight distribution is plotted, for each fraction and the trend plots were depicted for Mn, Mw, and PDI (FIG. 5). These trend plots are the actual comparison of the variations associated with each batch prepared. Using the reverse phase methodology the intact masses are checked for the trend lines of the masses when seen with IMMS (FIG. 6). These depict the conformational differences within the same masses. The comparison is done with the software to identify the variations with RLD (FIG. 7). This study can also be extrapolated and explored with the fraction masses.

In one embodiment, the present invention relates to statistical methods for characterizing and classifying Glatiramer acetate.

The statistical methods used herein include, but not limited to, normal distribution calculated on the basis of the mean and standard deviations of the list of masses obtained from the de-convolution.

The term “reference listed drug (RLD)” used herein refers to commercial sample of Copaxone® (Lot no: V01821 mainly and few other lots of Copaxone). RLD is used to establish equivalence of Glatiramer acetate analysed.

### EXAMPLES

The following examples are provided here to enable one skilled in the art to practice the invention and merely illustrate the process of this invention. However, it do not intended in any way to limit the scope of the present invention.

### Example 1

### Studying Six Proteins using Unifi and In-House Statistical Models (Covering Mass Range of 1 KDa to 66 KDa)

In this novel approach Unifi software version 1.8.2.169 from Waters has been used to process and deconvolute masses in HRMS (Waters Vion-IMS QTof). Six protein samples (four known commercial proteins and two synthetic purified peptides) were used as standards and analysed using the Unifi processing tool and in-house approach of applying various statistical models covering a mass range from 1 KDa to 66 KDa to check the overall accuracy using this novel approach. The proteins used in this test were Bovine Albumin (66 KDa), Bovine Carbonic anhydrase (29 KDa), Equine Cytochrome C (12 KDa), Bovine Apoprotein (6.5 KDa), Synthetic Teriparatide (4.1 KDa) and Synthetic Icatibant acetate (1.3 KDa).

Some of the chromatographic parameters:
Column: Acquity UPLC-BEH C18; 1.7 μm 2.1'3150 mm
Sample temperature: 4° C.
LC-UV detector wavelength: 280 nm
Flow rate: 0.250 ml/min
Injection volume: 4 μl
Run time: 60 min
Elution: Gradient
Mass range: 300-2000 m/z
Collision energy (low): 6 eV

The masses generated for the six proteins using Unifi is detailed in Table 1. The mass determination of few Copaxone lots and Test samples using Unifi and the novel statistical model are given in Table 2 and depicted in FIG. 6

TABLE 1
Mass analysis of some standard proteins using Unifi: masses observed
in Unifi vs. available sources (mass range of 1 KDa to 66 KDa)
Comparison of mass observed (Da) for various standard
proteins using UNIFI Vs. other available sources

| | | Theoretical | Most abundant mass |
|---|---|---|---|
| | | mass (Da) | observed in HRMS |
| Sr. | | (Drug bank/ | usingUnifi & in- |
| No. | Name of protein | other sources) | house protocols |
| 1 | Synthetic Icatibant acetate | 1304.5 | 1304.6 |
| 2 | Synthetic Teriparatide | 4117.7 | 4117.1 |
| 3 | Aprotinin (Bovine) | 6511.4 | 6523.0 |
| 4 | Cytochrome C (Equine) | 12384 | 12350.3 |
| 5 | Carbonic anhydrase | 29000 | 29006.0 |
| | (Bovine) | | |
| 6 | Albumin (Bovine) | 66463.0 | 66427.0 |

TABLE 2
Mass Determination of GMA in Copaxone and Test
samples using Unifi and novel statistical models
Mass Determination of GMA using Unifi and novel statistical
models in different lots of Copaxone and Test batches

| Sample Info | Lot no. | Mean mass |
|---|---|---|
| Copaxone (RED) | Copaxone_40 mg_V01821 | 7761 |
| lots | Copaxone_40 mg_C42277 | 7487 |
| | Copaxone_40 mg_V02371 | 7729 |
| | Copaxone_40 mg_V02661 | 7767 |
| | Copaxone_20 mg_P63291 | 7386 |
| | Copaxone_20 mg_P63304 | 7323 |
| Glatiramer acetate | GLAT/030/040 | 7690 |
| In-house batches | GLAT/001/044 | 7617 |
| | GLAT/001/084 | 7198 |
| | GLAT/030/043 | 7508 |
| | GLAT/030/044 | 7624 |
| | GLAT/030/047 | 7559 |

### Example 2

### Fractionation & Collection by Size Exclusion chromatography

Glatiramer acetate was run in a size exclusion chromatography mode in a UPLC coupled to HRMS (Waters Vion-IMS QTof), equipped with a UV visible detector, tertiary gradient pump, auto injector, Ion mobility separator, Quadrupole & Tof analyzers and Unifi software version 1.8.2.169. The fractions were collected at pre-optimized time points during the run time.

### Chromatographic Parameters:

Column ACQUITY UPLC Protein SEC BEH (4.6×300 mm; 1.7 μm)
Sample temperature: 10 ° C.
LC-UV detector wavelength: 280 nm
Flow rate: 0.250 ml/min
Diluent: MilliQ water
Injection volume: 1 μl
Run time: 30 min
Elution: Isocratic
   The collected fractions were further run in HRMS mode in the same instrument for data acquisition for subsequent stages of this experimental series.

### Example 3

### Data Acquisition and Processing using Unifi

The MS spectra obtained from the fraction analysis done on HRMS (Waters Vion-IMS QTof) was further processed and segregated by using Unifi software version 1.8.2.169 from Waters. Unifi is equipped with a processing tool which deconvolutes the MS spectra, by taking into account, the possible charges formed for a peptide/protein molecule, while acquiring data using an ESI mode of HRMS and returns with values very close to the actual mass of the molecular ions. The optimized parameters used for the processing tool are:

### Processing Parameters:

a) Maximum charge: 14
b) Input mass range (m/z): 300-1500
c) Output mass range (Da): 2000-25000
d) Resolution: Specific to resolution observed for the standard

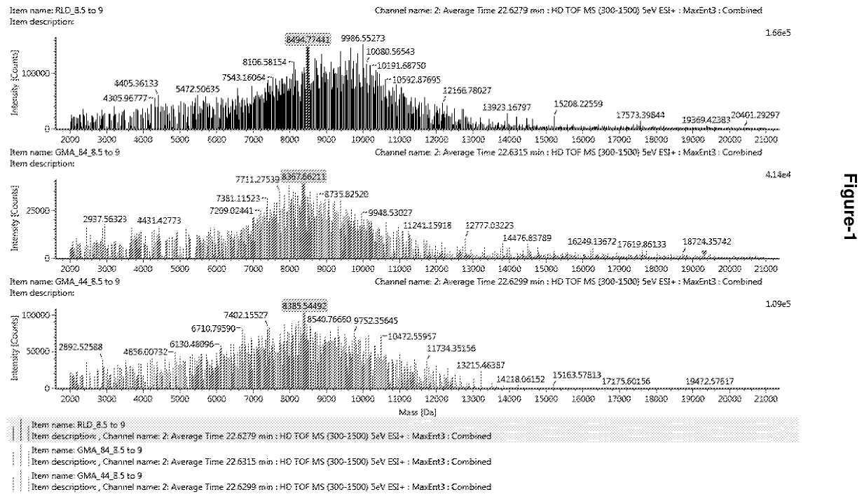

Spectra generated after the processing shows the deconvoluted masses for a specific fraction collected at a specific time point, presenting the mass distribution of Glatiramer acetate having masses in the range of 2000 Da-25000 Da in the X-axis and their corresponding intensities in the Y-axis (FIG. 1).

### Example 4

### Normal Distribution and Other Statistical Models for Characterization

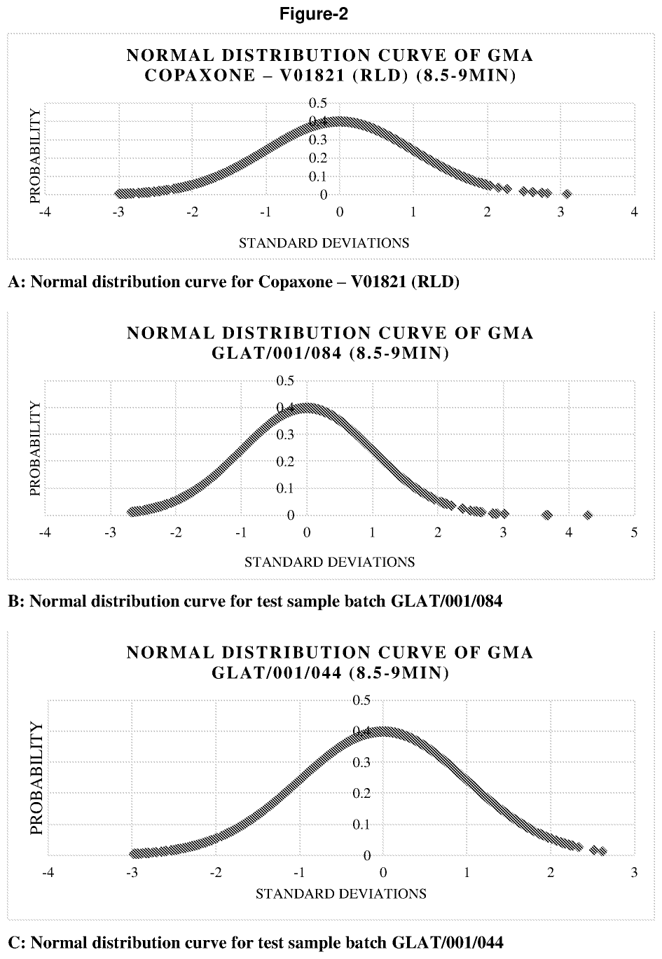

Using the deconvoluted normalized data obtained after processing, the entire data set is subjected to calculation of mean, standard deviations, and Z-score in order to obtain the normal distribution value which further provides the Gaussian or the Normal distribution curves (FIG. 2).

The mean, +1 and −1 standard deviations have been calculated by two statistical methods, first by calculating fractions individually and second by calculating together. The comparison of these methods of calculations for GMA mass distributions for test samples and RLD have been performed (Table 3-5).

TABLE 3
RLD: Comparison of different methods of
calculations for GMA mass distribution.
RLD (Copaxone)

| 12 fragments calculated | | 12 fragments calculated | | Full elution | |
|---|---|---|---|---|---|
| individually | | together | | 6.5 - 13.5 mins | |
| Mean | 7761 | Mean | 7504.33 | Mean | 8809.81 |
| Std. dev | 2361 | Std. dev | 3049.74 | Std. dev | 2505.31 |
| −3 | 14845 | −3 | 16653.54 | −3 | 16325.74 |
| −2 | 12484 | −2 | 13603.81 | −2 | 13820.43 |
| −1 | 10122 | −1 | 10554.07 | −1 | 11315.12 |
| 0 | 7761 | 0 | 7504.33 | 0 | 8809.81 |
| 1 | 5400 | 1 | 4454.59 | 1 | 6304.50 |
| 2 | 3038 | 2 | 1404.86 | 2 | 3799.19 |
| 3 | 677 | 3 | −1644.88 | 3 | 1293.88 |

TABLE 4
Test sample batch 84: Comparison of different methods
of calculations for GMA mass distribution
GLAT/001/084

| | |
|---|---|
| 12 fragments calculated | 12 fragments calculated |
| individually | together |

| Mean | 7197.86 | Mean | 7330.98 |
|---|---|---|---|
| Std. dev | 3284.50 | Std. dev | 3258.15 |
| −3 | 17051.35 | −3 | 17105.44 |
| −2 | 13766.86 | −2 | 13847.29 |
| −1 | 10482.36 | −1 | 10589.13 |
| 0 | 7197.86 | 0 | 7330.98 |
| 1 | 3913.37 | 1 | 4072.83 |
| 2 | 628.87 | 2 | 814.67 |
| 3 | −2655.62 | 3 | −2443.48 |

TABLE 5
Test sample batch 44: Comparison of different methods
of calculations for GMA mass distribution
GLAT/001/044

| | |
|---|---|
| 12 fragments calculated | 12 fragments calculated |
| individually | together |

| Mean | 7617.34 | Mean | 7716.57 |
|---|---|---|---|
| Std. dev | 3153.58 | Std. dev | 3103.00 |
| −3 | 17078.07 | −3 | 17025.57 |
| −2 | 13924.50 | −2 | 13922.57 |
| −1 | 10770.92 | −1 | 10819.57 |
| 0 | 7617.34 | 0 | 7716.57 |
| 1 | 4463.77 | 1 | 4613.57 |
| 2 | 1310.19 | 2 | 1510.57 |
| 3 | −1843.39 | 3 | −1592.43 |

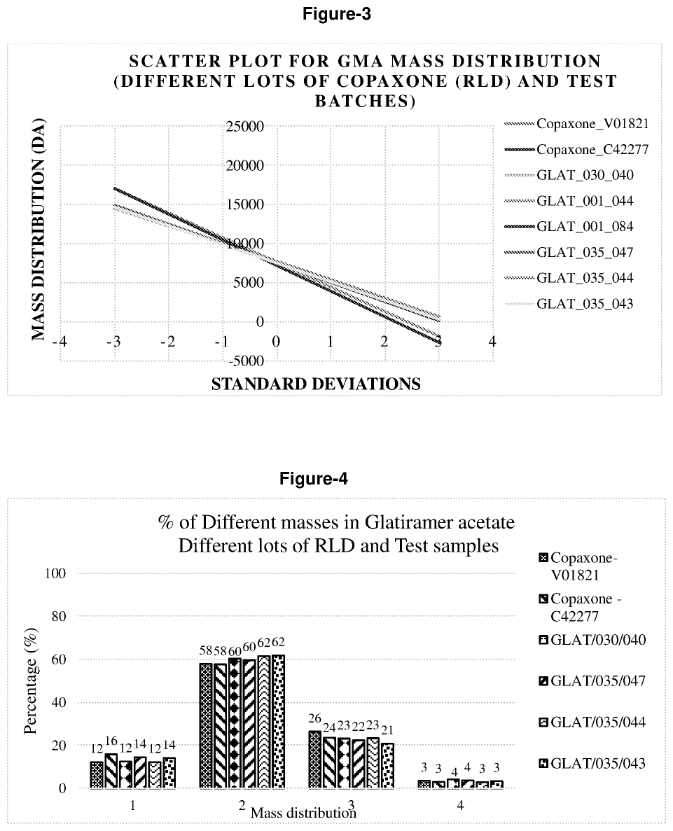

The standard deviation of the mass distribution observed in the RLD sample has been compared with the test sample batches (84 and 44) calculated based on the above tables and a scatter plot is plotted, which shows minimal variation in mass distribution (FIG. 3).

Another statistical method has been used to check the overall mass distribution of Glatiramer, wherein, the entire mass list (list of mass having the masses of all the molecular ions observed in the data set of a single injection) of GMA has been segregated into four categories for better characterization. The categories are based upon the mass range as follows:
Category I: Masses from 2000 Da -5000 Da
Category II: Masses from 5000 Da-9000 Da
Category III : Masses from 9000 Da-12,000 Da
Category IV Masses from 12,000 Da-20,000 Da

Samples from RLD and test sample batches have been analyzed as per the above categorization. The resultant data is represented as a percentage of the whole mass list for each sample (Table 6). A column chart has been used to compare the results of segregated masses observed in RLD and test sample batches (FIG. 4).

TABLE 6
Percentage of different masses in the entire GMA mass distribution
Percentage of different masses in the entire GMA mass distribution
(for different lots of Copaxone and Test batches)

| | 2000-5000 | 5000-9000 | 9000-12000 | 12000-20000 |
|---|---|---|---|---|
| Sample name | Da (%) | Da (%) | Da (%) | Da (%) |
| Copaxone- RLD- | 12 | 58 | 26 | 3 |
| V01821 | | | | |
| Copaxone - RLD - | 16 | 58 | 24 | 3 |
| C42277 | | | | |
| GLAT/030/040 | 12 | 60 | 23 | 4 |
| GLAT/035/047 | 14 | 60 | 22 | 4 |
| GLAT/035/044 | 12 | 62 | 23 | 3 |
| GLAT/035/043 | 14 | 62 | 21 | 3 |

Furthermore, the polymeric mass distribution values like Mn (Number average molecular weight), Mw (Weight average molecular weight) and Polydispersity index (PDI), which are already known from existing literatures, have also been calculated in order to confirm the observations of the statistical models of the present invention. The values obtained in test sample batches were compared with the RLD (Table 5). These values have been also confirmed by using the gas permeation chromatography software in a SEC-UPLC instrument coupled to a UV detector (Table 6).

TABLE 7
Comparison of polymeric mass distribution
values (RLD and test sample batches).

| Sr. No. | Sample name | Mn | Mw | PDI | Mz |
|---|---|---|---|---|---|
| 1 | Copaxone - V01821 | 7681 | 8290 | 1.08 | 8809 |
| 2 | Copaxone - C42277 | 7419 | 8097 | 1.09 | 8666 |
| 3 | GLAT/030/040 | 7601 | 8235 | 1.08 | 8798 |
| 4 | GLAT/035/043 | 7374 | 8014 | 1.09 | 8601 |
| 5 | GLAT/035/044 | 7529 | 8123 | 1.08 | 8639 |
| 6 | GLAT/035/047 | 7457 | 8129 | 1.09 | 8716 |

TABLE 8
Molecular weight distribution with
SEC chromatography and UV detection

| Sr. No. | Name of the Sample | Mn | Mw | PDI |
|---|---|---|---|---|
| 1 | Copaxone - V01821 | 7783 | 7803 | 1.002620 |
| 2 | GLAT/001/044 | 7763 | 7783 | 1.002546 |
| 3 | GLAT/001/084 | 7707 | 7723 | 1.001972 |

Lastly, in order to check the limits that has been observed for various mass distribution parameters obtained through this novel approach of characterizing Glatiramer acetate, through HRMS followed by using Unifi and in-house approach of applying various statistical models, extremities(mean mass range) for six parameters has been tabulated for different lots of Copaxone and test samples in Tables 9 to 14 and depicted in FIG. 7.

TABLE 9
Observed Extremity in Mean Mass of GMA
(Different lots of Copaxone and Test samples)
MEAN MASS

| Sr. No. | Lot no | Mean |
|---|---|---|
| 1 | Copaxone_V01821 | 7761 |
| 2 | Copaxone_C42277 | 7487 |
| 3 | Copaxone_V02371 | 7729 |
| 4 | Copaxone_V02661 | 7767 |
| 5 | Copaxone_P63291 | 7386 |
| 6 | Copaxone_P63304 | 7323 |
| 7 | GLAT/030/040 | 7690 |
| 8 | GLAT/035/043 | 7508 |
| 9 | GLAT/035/044 | 7624 |
| 10 | GLAT/035/047 | 7559 |

TABLE 10
Observed Extremity in standard deviation from mean mass
of GMA (Different lots of Copaxone and Test samples)
STANDARD DEVIATION OF MASS

| Sr. No. | Lot number | Std. dev |
|---|---|---|
| 1 | Copaxone_V01821 | 2361 |
| 2 | Copaxone_C42277 | 2449 |
| 3 | Copaxone_V02371 | 2470 |
| 4 | Copaxone_V02661 | 2507 |
| 5 | Copaxone_P63291 | 2538 |
| 6 | Copaxone_P63304 | 2491 |
| 7 | GLAT/030/040 | 2427 |
| 8 | GLAT/035/043 | 2392 |
| 9 | GLAT/035/044 | 2314 |
| 10 | GLAT/035/047 | 2439 |

TABLE 11
Observed Extremity in PDI of GMA (Different
lots of Copaxone and Test samples)
POLYDISPERSITY INDEX (PDI)

| Sr. No. | Lot number | PDI |
|---|---|---|
| 1 | Copaxone_V01821 | 1.08 |
| 2 | Copaxone_C42277 | 1.09 |
| 3 | Copaxone_V02371 | 1.09 |
| 4 | Copaxone_V02661 | 1.09 |
| 5 | Copaxone_P63291 | 1.10 |
| 6 | Copaxone_P63304 | 1.11 |
| 7 | GLAT/030/040 | 1.08 |
| 8 | GLAT/035/043 | 1.09 |
| 9 | GLAT/035/044 | 1.08 |
| 10 | GLAT/035/047 | 1.09 |

TABLE 12
Observed Extremity in Mw of GMA (Different
lots of Copaxone and Test samples)
WEIGHT AVERAGE MOLECULAR WEIGHT (Mw)

| Sr. No. | Lot number | Mw |
|---|---|---|
| 1 | Copaxone_V01821 | 8290 |
| 2 | Copaxone_C42277 | 8097 |
| 3 | Copaxone_V02371 | 8272 |
| 4 | Copaxone_V02661 | 8278 |
| 5 | Copaxone_P63291 | 7937 |
| 6 | Copaxone_P63304 | 7940 |
| 7 | GLAT/030/040 | 8235 |
| 8 | GLAT/035/043 | 8014 |
| 9 | GLAT/035/044 | 8123 |
| 10 | GLAT/035/047 | 8129 |

TABLE 13
Observed Extremity in Mn (Different lots of Copaxone and Test samples)
NUMBER AVERAGE MOLECULAR WEIGHT (Mn)

| Sr. No. | Lot number | Mn |
|---|---|---|
| 1 | Copaxone_V01821 | 7681 |
| 2 | Copaxone_C42277 | 7419 |
| 3 | Copaxone_V02371 | 7578 |
| 4 | Copaxone_V02661 | 7576 |
| 5 | Copaxone_P63291 | 7219 |
| 6 | Copaxone_P63304 | 7183 |
| 7 | GLAT/030/040 | 7601 |
| 8 | GLAT/035/043 | 7374 |
| 9 | GLAT/035/044 | 7529 |
| 10 | GLAT/035/047 | 7457 |

TABLE 14
Observed Extremity in Mz of GMA (Different
lots of Copaxone and Test samples)
Z AVERAGE MOLECULAR WEIGHT (Mz)

| Sr. No. | Lot number | Mz |
|---|---|---|
| 1 | Copaxone_V01821 | 8809 |
| 2 | Copaxone_C42277 | 8666 |
| 3 | Copaxone_V02371 | 8874 |
| 4 | Copaxone_V02661 | 8894 |
| 5 | Copaxone_P63291 | 8566 |
| 6 | Copaxone_P63304 | 8593 |
| 7 | GLAT/030/040 | 8798 |
| 8 | GLAT/035/043 | 8601 |
| 9 | GLAT/035/044 | 8639 |
| 10 | GLAT/035/047 | 8716 |

1. A process for characterizing of Glatiramer acetate, the process comprising:
a. subjecting a sample of Glatiramer acetate to size exclusion chromatography (SEC);
b. separating various fractions based on its size and subjecting the separated fractions through reverse phase chromatography to HRMS;
c. processing of electrospray ionized ion masses and obtaining deconvoluted masses for checking mass distribution pattern of Glatiramer acetate; and
d. characterizing the Glatiramer acetate using a statistical model.

2. The process of claim 1, wherein the size exclusion chromatography is coupled with ultra-performance liquid chromatography and UV detector (SEC-UPLC-UV).

3. The process of claim 1, wherein electrospray ionized masses data are generated by tandem mass spectrometric data such as ESI-QTOF.

4. The process of claim 1, wherein step (c) is performed using Unifi software version 1.8.2.169 from Waters.

5. The process of claim 1, wherein characterizing the Glatiramer acetate involves used of statistical models for the determination of mean mass, standard deviations, distribution curves, +1 and −1 deviations and mass categorizations for characterization of Glatiramer acetate.

6. A process for characterization of mixture of polypetides, the process comprising using SEC-HRMS (Vion IMS-QTof) and statistical models for interpretations and illustrations wherein the mixture of polypetides consists of L-Alanine, L-Tyrosine, L-Lysine and L-Glutamic acid.
